(19) United States
(12) Patent Application Publication
(10) Pub. No.: US 2026/0232248 A1
(43) Pub. Date: Aug. 13, 2026

(54) TECHNIQUES FOR RECOMMENDING A WRIST-WEARABLE DEVICE POSITION FOR PHYSIOLOGICAL MEASUREMENTS BASED ON PHOTOPLETHYSMOGRAPHY (PPG) DATA AND SYSTEMS OF USE THEREOF

(71) Applicant: Meta Platforms Technologies, LLC, Menlo Park, CA (US)

(72) Inventors: Teodora Sandra Buda, Barcelona (ES); Kevin Kai-feng Chiou, Hawthorn Woods, IL (US); Michael John Toksvig, Palo Alto, CA (US)

(21) Appl. No.: 19/538,714

(22) Filed: Feb. 12, 2026

Related U.S. Application Data
(60) Provisional application No. 63/757,577, filed on Feb. 12, 2025.

(51) Int. Cl. A61B 5/332

(52) U.S. Cl. CPC A61B 5/332

(57) ABSTRACT

A method for recommending a wrist-wearable device position for physiological measurements based on photoplethysmography (PPG) data is described. The method includes, while the wrist-wearable device is at first and second positions: (i) receiving first and second PPG data captured at the one or more PPG sensors and first and second calibration PPG data captured at the one or more calibration PPG sensors, (ii) generating first and second physiological data based on the first and second PPG data and first and second calibration physiological data based on the and second first calibration PPG data, and (iii) determining first and second placement errors based on a comparison between the first and second PPG data and the first and second calibration PPG data. The method includes, in accordance with a determination that the first placement error is lesser, presenting a recommendation to the user to use the wrist-wearable device at the first position.

### RELATED APPLICATION

This application claims priority to U.S. Provisional Application Serial No. 63/757,577, filed February 12, 2025, entitled “Identification Of Optimal Wearable Device Setting For Physiological Metric Measurements,” which is incorporated herein by reference.

### TECHNICAL FIELD

This relates generally to calibrating physiological measurements based on photoplethysmography (PPG) data captured at a wrist-wearable device.

### BACKGROUND

Smart watches, fitness bracelets, smart rings, etc., are becoming increasingly popular. These devices include a variety of sensors that may be used to monitor various physiological metrics, such as heart rate, blood pressure, oxygen saturation, heart rate variability, etc. Wrist-wearable device fit is crucial for accurate measurement of these physiological metrics. However, it can be difficult for a user to identify an optimal setting or positioning for a wrist-wearable device. An optimal setting or positioning for the wrist-wearable device may be uncomfortable for the user, and comfortable setting or wrist-wearable device may lead to poor measurement of physiological metrics.

As such, there is a need to address one or more of the above-identified challenges. A brief summary of solutions to the issues noted above are described below.

### SUMMARY

One example of a method for recommending a wrist-wearable device position for physiological measurements based on photoplethysmography (PPG) data is described herein. This example method is executed at a system including a wrist-wearable device and a calibration device while the wrist-wearable device and the calibration device are worn by the user. The wrist-wearable device includes one or more PPG sensors, and the calibration device includes one or more calibration PPG sensors. The method includes, while the wrist-wearable device is at a first position on a wrist of the user (e.g., a first comfortable position with a first comfortable tightness):

(I) receiving first PPG data captured at the one or more PPG sensors and first calibration PPG data captured at the one or more calibration PPG sensors, (ii) generating first physiological data (e.g., blood pressure data predictions) based on the first PPG data and first calibration physiological data (e.g., calibration blood pressure data predictions) based on the first calibration PPG data, and (iii) determining a first placement error based on a comparison between the first PPG data and the first calibration PPG data. The method further includes, while the wrist-wearable device is at a second position, distinct from the first position, on the wrist of the user (e.g., a second comfortable position with a second comfortable tightness): (i) receiving second PPG data captured at the one or more PPG sensors and second calibration PPG data captured at the one or more calibration PPG sensors, generating second physiological data based on the second PPG data and second calibration physiological data based on the second calibration PPG data, and determining a second placement error based on a comparison between the second PPG data and the second calibration PPG data. The method further includes, in accordance with a determination that the first placement error is less than the second placement error, causing a recommendation to be presented to the user, the recommendation recommending that the user wear the wrist-wearable device at the first position on the wrist of the user.

Instructions that cause performance of the methods and operations described herein can be stored on a non-transitory computer readable storage medium. The non-transitory computer-readable storage medium can be included on a single electronic device or spread across multiple electronic devices of a system (computing system). A non-exhaustive of list of electronic devices that can either alone or in combination (e.g., a system) perform the method and operations described herein include an extended-reality (XR) headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIG. 1 illustrates a user wearing a wrist-wearable device and a calibration device, in accordance with some embodiments.

FIG. 2 illustrates a method for generating a recommendation of an optimal comfortable position and an optimal comfortable tightness for the user to wear the wrist-wearable device, in accordance with some embodiments.

FIG. 3 illustrates a flow diagram of a method for recommending a wrist-wearable device position for physiological measurements based on PPG data, in accordance with some embodiments.

FIGS. 4A, 4B, 4C-1,and 4C-2, illustrate example mixed-reality (MR) and augmented-reality (AR) systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user’s physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user’s hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user’s hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single- or double-finger tap on a table, on a user’s hand or another finger, on the user’s leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user’s possession (e.g., smartphones, laptops, imaging devices, intermediary devices, and/or other devices described herein).

The input modalities as alluded to above can be varied and are dependent on a user’s experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors (used interchangeably with neuromuscular-signal sensors); (iii) IMUs for detecting, for example, angular rate, force, magnetic field, and/or changes in acceleration; (iv) heart rate sensors for measuring a user’s heart rate; (v) peripheral oxygen saturation (SpO₂) sensors for measuring blood oxygen saturation and/or other biometric data of a user; (vi) capacitive sensors for detecting changes in potential at a portion of a user’s body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user’s environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

### Recommending A Wrist-Wearable Device Position For Physiological Measurements Based On Photoplethysmography (PPG) Data

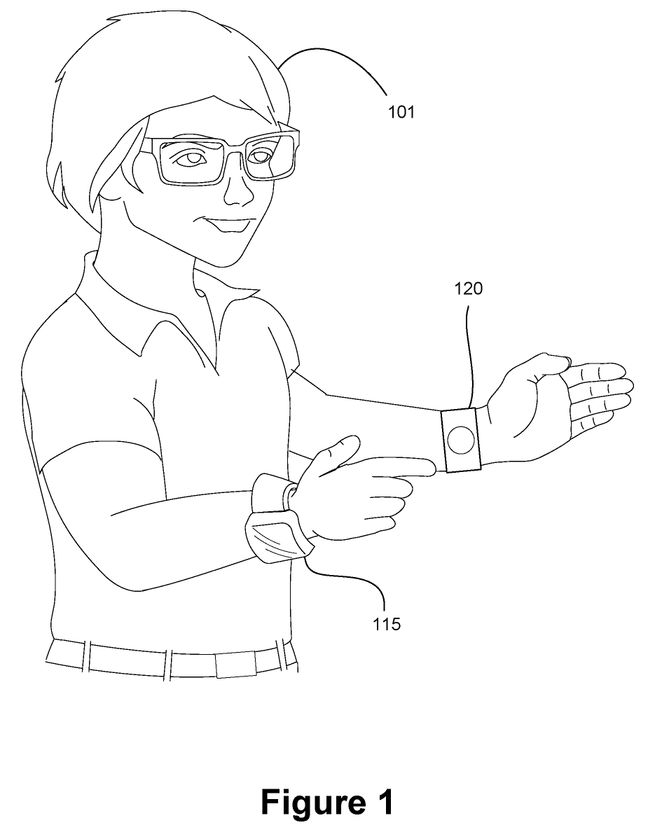

FIG. 1 illustrates a user 101 wearing a wrist-wearable device 115 and a calibration device 120, in accordance with some embodiments. The wrist-wearable device 115 is one or more of a smart watch (e.g., as illustrated in FIG. 1), a fitness band, a smart arm band, and/or another wrist/forearm-wearable device that includes one or more sensors. In some embodiments, the techniques and systems described herein include another wearable device (e.g., a head-wearable smart device, a leg-wearable smart device, a body-integrated device, etc.) with one or more sensors that performs the same operations of the wrist-wearable device 115. The wrist-wearable device 115 includes one or more photoplethysmography (PPG) sensors (and/or one or more other photodetectors) for capturing PPG data from a wrist/forearm of the user 101 while the wrist-wearable device 115 is worn by the user 101. The PPG data is used to determine one or more physiological datum including one or more of blood pressure data, heart rate data, blood oxygen saturation data, heart rate variability data, and/or respiration rate data. In some embodiments, the wrist-wearable device 115 includes one or more other sensors for capturing other data (e.g., one or more biopotential sensors (e.g., one or more electromyography (EMG) sensors), one or more inertial measurement unit (IMU) sensors, one or more microphones, etc.). In some embodiments, the calibration device 120 is another wrist-wearable device. In some embodiments, the calibration device 120 is another wrist-wearable device of a same device type as the wrist-wearable device 115 (e.g., the wrist-wearable device 115 and the calibration device 120 are both smart watches). The calibration device 120 also includes one or more calibration PPG sensors (and/or one or more other calibration photodetectors) for capturing calibration PPG data from another wrist/forearm of the user 101 while the wrist-wearable device 115 is worn by the user 101. As an example illustrated in FIG. 1, the user 101 wears the wrist-wearable device 115 on their right wrist and the calibration device 120 on their left wrist. In some embodiments, the calibration device 120 is communicatively coupled to the wrist-wearable device 115, and/or the calibration device 120 and the wrist-wearable device 115 are both communicatively coupled to an intermediary device (e.g., a smartphone, a computer, a server, etc.), and the calibration device 120 and the wrist-wearable device 115 share the PPG data and the calibration PPG data with each other and the intermediary device.

The wrist-wearable device 115 and the calibration device 120 each include a respective wristband that affix the wrist-wearable device 115 and/or the calibration device 120 to a respective wrist/forearm of the user 101. In some embodiments, the one or more PPG sensors and the one or more calibration PPG sensors are position on the wristband of the wrist-wearable device 115 and the calibration device 120, respectively. A position of the wrist-wearable device 115 and/or the calibration device 120 on a wrist/forearm of the user 101 effects an accuracy of respective PPG data captured at the one or more PPG sensors and/or the one or more calibration PPG sensors. Additionally, a tightness of the respective wristband of the wrist-wearable device 115 and/or the calibration device 120 on a wrist/forearm of the user 101 effects an accuracy of respective PPG data captured at the one or more PPG sensors and/or the one or more calibration PPG sensors. While an optimal position of the wrist-wearable device 115 and/or the calibration device 120 and an optimal tightness of the respective wristband for capturing most accurate PPG data may vary among a plurality of users, generally the optimal position of the wrist-wearable device 115 and/or the calibration device 120 is two finger-widths above the respective wrist of the user 101, and the optimal tightness of the respective wristband is as tight as possible without causing harm to the user 101. While this may be the optimal position and the optimal tightness for capturing the most accurate PPG data, this is most often not an optimal position and/or an optimal tightness of the wrist-wearable device 115 for the comfort of the user 101 wearing the wrist-wearable device 115 in everyday settings.

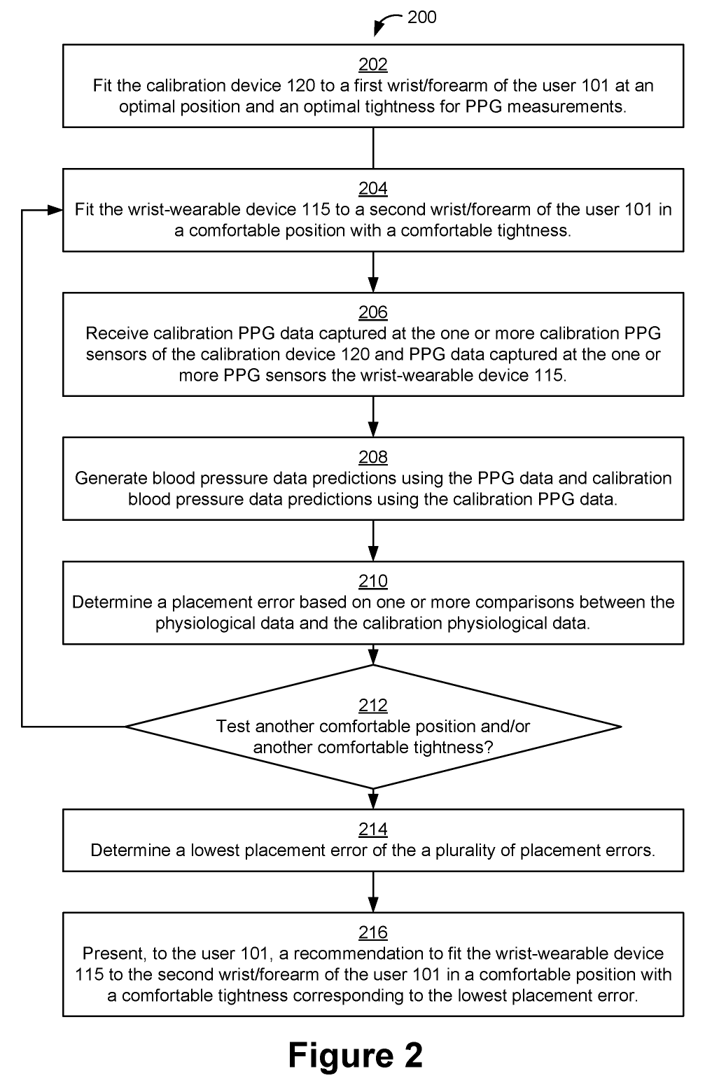

FIG. 2 illustrates a method 200 for generating a recommendation of an optimal comfortable position and an optimal comfortable tightness for the user 101 to wear the wrist-wearable device 115, in accordance with some embodiments. In some embodiments, the method 200 is performed at one or more processors communicatively coupled to the wrist-wearable device 115 and the calibration device 120 (e.g., one or more processors of the wrist-wearable device 115, one or more processors of the calibration device 120, and/or one or more processors of the intermediary device). The method 200 includes the user 101 fitting the calibration device 120 to a first wrist/forearm (e.g., the left wrist, as illustrated in FIG. 1) of the user 101 in the optimal position with the optimal tightness for PPG measurements (e.g., the optimal position is the calibration device 120 is positioned two finger-widths above the respective wrist of the user 101, and the optimal tightness of the respective wristband is as tight as possible without causing harm to the user 101) (202). The method 200 further includes the user 101 fitting the wrist-wearable device 115 to a second wrist/forearm (e.g., the right wrist, as illustrated in FIG. 1) of the user 101 in a first comfortable position with a first comfortable tightness (e.g., a position and a tightness that the user 101 could comfortably wear the wrist-wearable device 115 for an extended period of time in everyday settings) (204). In some embodiments, step 204 may be performed before step 202 and/or step 204 and step 202 may be performed simultaneously. In some embodiments, one or more instructions (e.g., “Please place the calibration band on your dominant wrist positioned two finger-widths the wrist and as tight as possible without causing harm. Then place the watch on your non-dominant wrist in position that is comfortable for you.”) are presented to the user 101 (e.g., at the wrist-wearable device 115, the calibration device 120, and/or the intermediary device) instructing the user 101 on performing step 202 and/or step 204.

The method 200 further includes, while the wrist-wearable device 115 is in the first comfortable position with the first comfortable tightness, receiving first PPG data captured at the one or more PPG sensors the wrist-wearable device 115 and first calibration PPG data captured at the one or more calibration PPG sensors of the calibration device 120 (206). In some embodiments, the first PPG data and the first calibration PPG data are captured synchronously. In some embodiments, the wrist-wearable device 115 includes one or more pressure sensors that measure the first comfortable tightness. The method 200 further includes generating first physiological data (e.g., blood pressure data predictions) using the first PPG data and first calibration physiological data (e.g., calibration blood pressure data predictions) using the first calibration PPG data (208). In some embodiments, the first physiological data and the first calibration physiological data are determined using one or more pre-trained models (e.g., a model trained on one or more comparisons between known PPG data and known physiological data). In some embodiments, the one or more pre-trained models include one or more artificial intelligence (AI) models and/or one or more machine-learning (ML) models. The method 200 further includes determining a first placement error based on one or more comparisons between the first physiological data and the first calibration physiological data (210). The first placement error represents a degree of accuracy of the first physiological data based on a disparity between the first physiological data and the first calibration physiological data.

The method 200 continues by repeating steps 204-210. The method 200 further includes the user 101 fitting the wrist-wearable device 115 to the second wrist/forearm of the user 101 in a second comfortable position with a second comfortable tightness (distinct from the first comfortable position and/or distinct from the first comfortable tightness) (e.g., a different position and/or a different tightness that the user 101 could comfortably wear the wrist-wearable device 115 for an extended period of time in everyday settings) (204). The method 200 further includes, while the wrist-wearable device 115 is in the second comfortable position with the second comfortable tightness, receiving second PPG data captured at the one or more PPG sensors the wrist-wearable device 115 and second calibration PPG data captured at the one or more calibration PPG sensors of the calibration device 120 (206). In some embodiments, the second PPG data and the second calibration PPG data are captured synchronously. In some embodiments, the one or more pressure sensors of the wrist-wearable device 115 measure the second comfortable tightness. The method 200 further includes generating second physiological data (e.g., second blood pressure data predictions) using the second PPG data and second calibration physiological data (e.g., second calibration blood pressure data predictions) using the second calibration PPG data (208). In some embodiments, the second physiological data and the second calibration physiological data are determined using the one or more pre-trained models (e.g., one or more pre-trained models personalized to the user 101 based on PPG morphology, wrist-circumference, skin-tone, sex, other demographic, etc.). The method 200 further includes determining a second placement error based on one or more comparisons between the second physiological data and the second calibration physiological data (210). The second placement error represents a degree of accuracy of the second physiological data based on a disparity between the second physiological data and the second calibration physiological data.

In some embodiments, the method 200 continues by reposition the wrist-wearable device 115 to repeat steps 204-210 one or more times with the wrist-wearable device 115 fitted at one or more further comfortable positions with one or more further comfortable tightnesses (distinct from the first comfortable position and/or the second comfortable position, and/or distinct from the first comfortable tightness and/or the second comfortable tightness). In some embodiments, the user 101 may choose to reposition the wrist-wearable device 115 to continue repeating steps 204-210 as many times as the user 101 wishes with the wrist-wearable device 115 fitted at the one or more further comfortable positions with the one or more further comfortable tightnesses (212). In some embodiments, a request (e.g., “Okay, now try moving your watch to another comfortable position on your wrist or adjust the tightness of the wristband.”) may be presented (e.g., at the wrist-wearable device 115, the calibration device 120, and/or the intermediary device) to the user 101 requesting that the user 101 reposition the wrist-wearable device 115 to repeat steps 204-210 a predetermined number of times (e.g., seven times to determine seven placement errors corresponding to seven distinct comfortable positions and/or comfortable tightnesses). In some embodiments, another request (e.g., “Calibration failed, please try moving your watch to another position or tightening the wristband.”) may be presented to the user 101 requesting that the user 101 repeat steps 204-210 until a respective placement error (corresponding to a respective comfortable positions and a respective comfortable tightnesses) is below an error threshold.

After the user 101 repositions the wrist-wearable device 115 for a plurality times to complete steps 204-210 for a plurality of times, a plurality of placement errors (e.g., the first placement error, the second placement error, a third placement error, etc.) is obtained. Each placement error of the plurality of placement errors is associated with a respective comfortable position with a respective comfortable tightnesses of a plurality of comfortable positions with comfortable tightnesses (e.g., the first comfortable position with the first comfortable tightness, the second comfortable position with the second comfortable tightness, a third comfortable position with a third comfortable tightness, etc.). The method 200 further includes determining a lowest placement error of the plurality of placement errors (214). The lowest placement error is representative of a respective physiological data that is most accurate based on a disparity between the respective physiological data and its corresponding calibration physiological data. The method 200 further includes presenting, to the user 101, a recommendation (e.g., “Okay, let’s wear the watch in the second position you tried for the best results.”) to fit the wrist-wearable device 115 to the second wrist/forearm of the user 101 in a comfortable position with a comfortable tightness corresponding to the lowest placement error (216). For example, if the second placement error is less than the first placement error (indicating that the second physiological data is more accurate than the first physiological data), a recommendation to fit the wrist-wearable device 115 to the second wrist/forearm of the user 101 in the second comfortable position with the second comfortable tightness is presented to the user 101. In some embodiments, the recommendation to fit the wrist-wearable device 115 to the second wrist/forearm of the user 101 in a comfortable position with a comfortable tightness corresponding to the lowest placement error is a visual recommendation (e.g., a visual notification presented at one or more displays of the wrist-wearable device 115, the calibration device 120, and/or the intermediary device) and/or an audio recommendation (e.g., an audio notification presented at one or more speakers of the wrist-wearable device 115, the calibration device 120, and/or the intermediary device).

After the user 101 fits the wrist-wearable device 115 to the second wrist/forearm of the user 101 in the comfortable position with the comfortable tightness corresponding to the lowest placement error, user 101 may remove the calibration device 120 and utilize the wrist-wearable device 115 to capture additional PPG data and generate additional physiological data based on the additional PPG data. The additional physiological data may be utilized by one or more programs and/or applications (e.g., a health application, a fitness application, a medical application, etc.) executed at the wrist-wearable device 115 and/or another device (e.g., a head-wearable device, a smartphone, a computer, etc.) communicatively coupled to the wrist-wearable device 115. For example, the one or more PPG sensors of the wrist-wearable device 115 may captured the additional PPG data, which is used to generate additional blood pressure data. The additional blood pressure data is then presented within a user interface of a fitness-tracking application displayed at one or more displays of a smartphone communicatively coupled to the wrist-wearable device 115.

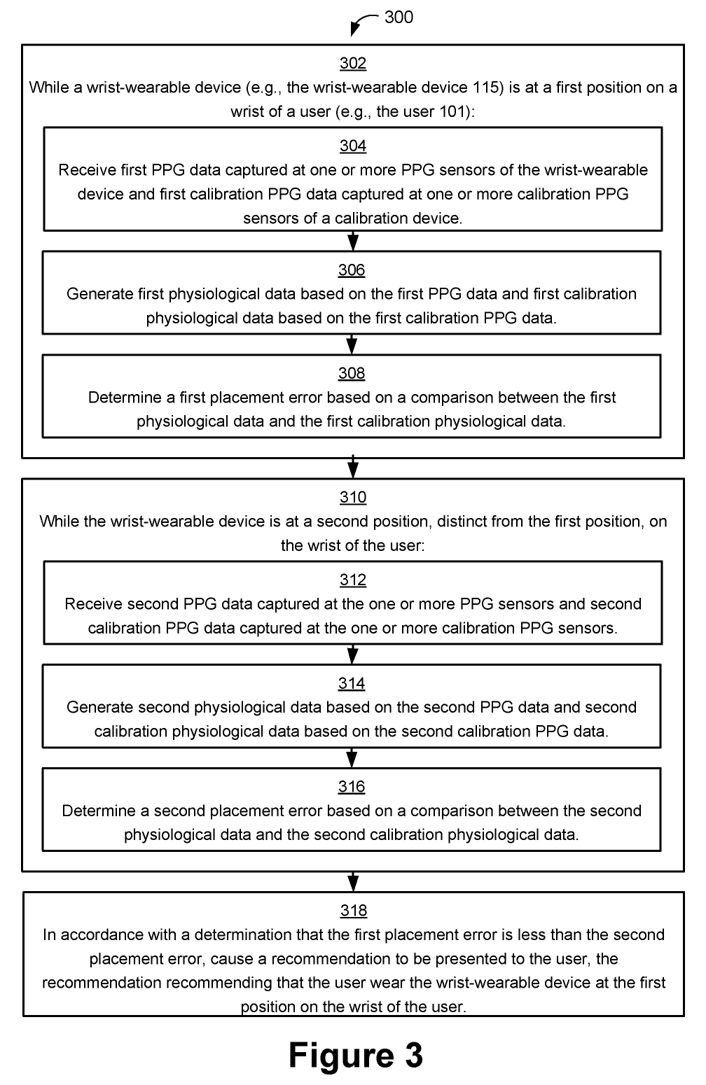

FIG. 3 illustrates a flow diagram of a method 300 for recommending a wrist-wearable device position for physiological measurements based on PPG data, in accordance with some embodiments. Operations (e.g., steps) of the method 300 can be performed by one or more processors (e.g., central processing unit and/or MCU) of a system including a wrist-wearable device, a calibration device, and/or an intermediary device. At least some of the operations shown in FIG. 3 correspond to instructions stored in a computer memory or computer-readable storage medium (e.g., storage, RAM, and/or memory) of the wrist-wearable device, the calibration device, and/or the intermediary device. Operations of the method 300 can be performed by a single device alone or in conjunction with one or more processors and/or hardware components of another communicatively coupled device and/or instructions stored in memory or computer-readable medium of the other device communicatively coupled to the system. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device, but should not be construed as limiting the performance of the operation to the particular device in all embodiments.

(A1) FIG. 3 shows a flow chart of a method 300 for recommending a wrist-wearable device position for physiological measurements based on PPG data, in accordance with some embodiments. The method 300 occurs while a wrist-wearable device (e.g., the wrist-wearable device 115) and a calibration device (e.g., the calibration device 120) are worn by a user (e.g., 101). The wrist-wearable device includes one or more PPG sensors, and the calibration device includes one or more calibration PPG sensors. The method 300 includes, while the wrist-wearable device is at a first position on a wrist of the user (e.g., the first comfortable position with the first comfortable tightness, as described in reference to FIG. 2) (302): (i) receiving first PPG data captured at the one or more PPG sensors and first calibration PPG data captured at the one or more calibration PPG sensors (304), (ii) generating first physiological data (e.g., blood pressure data predictions) based on the first PPG data and first calibration physiological data (e.g., calibration blood pressure data predictions) based on the first calibration PPG data (306), and (iii) determining a first placement error based on a comparison between the first PPG data and the first calibration PPG data (308). The method 300 further includes, while the wrist-wearable device is at a second position, distinct from the first position, on the wrist of the user (e.g., the second comfortable position with the second comfortable tightness, as described in reference to FIG. 2) (310): (i) receiving second PPG data captured at the one or more PPG sensors and second calibration PPG data captured at the one or more calibration PPG sensors (312), generating second physiological data based on the second PPG data and second calibration physiological data based on the second calibration PPG data (314), and determining a second placement error based on a comparison between the second PPG data and the second calibration PPG data (316). The method 300 further includes, in accordance with a determination that the first placement error is less than the second placement error, causing a recommendation to be presented to the user, the recommendation recommending that the user wear the wrist-wearable device at the first position on the wrist of the user (e.g., the recommendation to fit the wrist-wearable device 115 to the second wrist/forearm of the user 101 in a comfortable position with a comfortable tightness corresponding to the lowest placement error is a visual recommendation, as described in reference to FIG. 2) (318).

(A2) In some embodiments of A2, the method 300 further includes, while the wrist-wearable device is at a third position on the wrist of the user: (i) receiving third PPG data captured at the one or more PPG sensors and third calibration PPG data captured at the one or more calibration PPG sensors, (ii) generating third physiological data based on the third PPG data and third calibration physiological data based on the third calibration PPG data, and (iii) determining a third placement error based on a comparison between the third PPG data and the third calibration PPG data. The method 300 further includes, while the wrist-wearable device is at a fourth position, distinct from the third position, on the wrist of the user: (i) receiving fourth PPG data captured at the one or more PPG sensors and fourth calibration PPG data captured at the one or more calibration PPG sensors, (ii) generating fourth physiological data based on the fourth PPG data and fourth calibration physiological data based on the fourth calibration PPG data, and (iii) determining a fourth placement error based on a comparison between the fourth PPG data and the fourth PPG data. The method 300 further includes, while the wrist-wearable device is at a fifth position, distinct from the third position and the fourth position, on the wrist of the user: (i) receiving fifth PPG data captured at the one or more PPG sensors and fifth calibration PPG data captured at the one or more calibration PPG sensors, (ii) generating fifth physiological data based on the fifth PPG data and fifth calibration physiological data based on the fifth calibration PPG data, and (iii) determining a fifth placement error based on a comparison between the fifth PPG data and the fifth PPG data. The method 300 further includes, in accordance with a determination that the fifth placement error is less than the third placement error and the fourth placement error, causing another recommendation to be presented to the user, the other recommendation recommending that the user wear the wrist-wearable device at the fifth position on the wrist of the user.

(A3) In some embodiments of any of A1-A2, the method 300 further includes, while the wrist-wearable device has a first tightness around the wrist of the user: (i) receiving sixth PPG data captured at the one or more PPG sensors and sixth calibration PPG data captured at the one or more calibration PPG sensors, (ii) generating sixth physiological data based on the sixth PPG data and sixth calibration physiological data based on the sixth calibration PPG data, and (iii) determining a sixth placement error based on a comparison between the sixth PPG data and the sixth calibration PPG data. The method 300 further includes, while the wrist-wearable device has a second tightness, distinct from the first tightness, around the wrist of the user: (i) receiving seventh PPG data captured at the one or more PPG sensors and seventh calibration PPG data captured at the one or more calibration PPG sensors, (ii) generating seventh physiological data based on the seventh PPG data and seventh calibration physiological data based on the seventh calibration PPG data, and (iii) determining a seventh placement error based on a comparison between the seventh PPG data and the seventh calibration PPG data. The method 300 further includes, in accordance with a determination that the seventh placement error is less than the sixth placement error, causing an additional recommendation to be presented to the user, the additional recommendation recommending that the user wear the wrist-wearable device with the second tightness around the wrist of the user.

(A4) In some embodiments of any of A1-A3, the method 300 further includes, while the wrist-wearable device is at a sixth position on the wrist of the user and has a third tightness around the wrist of the user: (i) receiving eighth PPG data captured at the one or more PPG sensors and eighth calibration PPG data captured at the one or more calibration PPG sensors, (ii) generating eighth physiological data based on the eighth PPG data and eighth calibration physiological data based on the eighth calibration PPG data, and (iii) determining an eighth placement error based on a comparison between the eighth PPG data and the eighth calibration PPG data. The method 300 further includes, while the wrist-wearable device is at a seventh position, distinct from the sixth position, on the wrist of the user and has a fourth tightness, distinct from the third tightness, around the wrist of the user: (i) receiving ninth PPG data captured at the one or more PPG sensors and ninth calibration PPG data captured at the one or more calibration PPG sensors, (ii) generating ninth physiological data based on the ninth PPG data and ninth calibration physiological data based on the ninth calibration PPG data, and (iii) determining a ninth placement error based on a comparison between the ninth PPG data and the ninth calibration PPG data. The method 300 further includes, in accordance with a determination that the eighth placement error is less than the ninth placement error, causing a further recommendation to be presented to the user, the further recommendation recommending that the user wear the wrist- wearable device at the seventh position on the wrist of the user and with the fourth tightness around the wrist of the user.

(A5) In some embodiments of any of A1-A4, the wrist-wearable device is worn on the wrist of the user (e.g., a right wrist of the user 101, as illustrated in FIG. 1), and the calibration device is worn on a second wrist , distinct from the wrist, of the user (e.g., a left wrist of the user 101, as illustrated in FIG. 1).

(A6) In some embodiments of any of A1-A5, the first position on the wrist on the user and the second position on the wrist of the user are positions that the user finds comfortable for wearing the wrist-wearable device. The calibration device is worn on at a position on the second wrist that is optimal for capturing PPG data.

(A7) In some embodiments of any of A1-A6, the method 300 further includes, before receiving the first PPG data and the first calibration PPG data, causing one or more instructions to be presented to the user, the one or more instructions instructing the user to: (i) fit the calibration device at the position on the second wrist that is optimal for capturing PPG data and (ii) fit the wrist-wearable device at a position on the wrist on the user that the user finds comfortable for wearing the wrist-wearable device.

(A8) In some embodiments of any of A1-A7, the method 300 further includes, before receiving the second PPG data and the second calibration PPG data, causing one or more additional instructions to be presented to the user, the one or more additional instructions instructing the user to fit the wrist-wearable device at another position on the wrist on the user that the user finds comfortable for wearing the wrist-wearable device.

(A9) In some embodiments of any of A1-A8, the first PPG data is captured at the one or more PPG sensors and the first calibration PPG data is captured at the one or more calibration PPG sensors simultaneously at a first point in time. The second PPG data is captured at the one or more PPG sensors and the second calibration PPG data is captured at the one or more calibration PPG sensors simultaneously at a second point in time, distinct from the first point in time.

(A10) In some embodiments of any of A1-A9, the first physiological data is generated from the first PPG data using one or more pre-trained models, the first calibration physiological data is generated from the first calibration PPG data using the one or more pre-trained models, the second physiological data is generated from the second PPG data using the one or more pre-trained models, and the second calibration physiological data is generated from the second calibration PPG data using the one or more pre-trained models.

(A11) In some embodiments of any of A1-A10, the one or more pre-trained models includes one or more of an artificial intelligence (AI) model and a machine-learning (ML) model.

(A12) In some embodiments of any of A1-A11, the first physiological data, the first calibration physiological data, the second physiological data, and the second calibration physiological data each include one or more of respective blood pressure data, respective heart rate data, respective blood oxygen saturation data, respective heart rate variability data, and/or respective respiration rate data.

(A13) In some embodiments of any of A1-A12, the recommendation is one or more of (i) a visual recommendation presented at one or more displays of one or more of the wrist-wearable device, the calibration device, and another device (e.g., the intermediary device, as described in reference to FIGS. 1-2) communicatively coupled to the wrist-wearable device and (ii) an audio recommendation presented at one or more speakers of one or more of the wrist-wearable device, the calibration device, and the other device.

(A14) In some embodiments of any of A1-A13, the wrist-wearable device is a smart watch.

(B1) In accordance with some embodiments, a system that includes a wrist wearable device (e.g., the wrist-wearable device 115) and a calibration device (e.g., the calibration device 120), and the system is configured to perform operations corresponding to any of A1-A14.

(C1) In accordance with some embodiments, a non-transitory computer readable storage medium including instructions that, when executed by one or more processors communicatively couped to a wrist wearable device (e.g., the wrist-wearable device 115) and a calibration device (e.g., the calibration device 120), cause the one or more processors to perform operations corresponding to any of A1-A14.

(D1) In accordance with some embodiments, a wrist-wearable device (e.g., the wrist-wearable device 115) communicatively coupled to a calibration device (e.g., the calibration device 120), and the wrist-wearable device is configured to perform operations that correspond to any of A1-A14.

(E1) In accordance with some embodiments, a calibration device (e.g., the calibration device 120) communicatively coupled to a wrist-wearable device (e.g., the wrist-wearable device 115), and the calibration device is configured to perform operations that correspond to any of A1-A14.

(F1) In accordance with some embodiments, an intermediary device is communicatively coupled to a calibration device (e.g., the calibration device 120) and a wrist-wearable device (e.g., the wrist-wearable device 115), and the intermediary device is configured to perform operations that correspond to any of A1-A14.

The devices described above are further detailed below, including wrist-wearable devices, headset devices, systems, and haptic feedback devices. Specific operations described above may occur as a result of specific hardware, such hardware is described in further detail below. The devices described below are not limiting and features on these devices can be removed or additional features can be added to these devices.

### Example Extended-Reality Systems

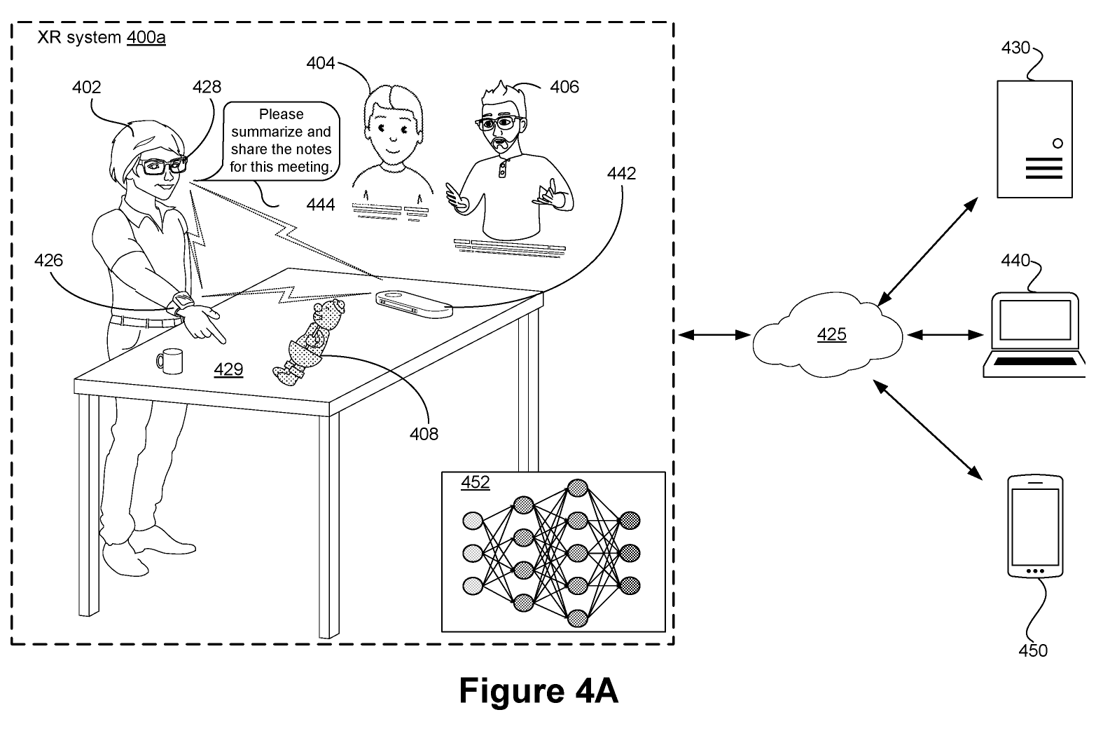

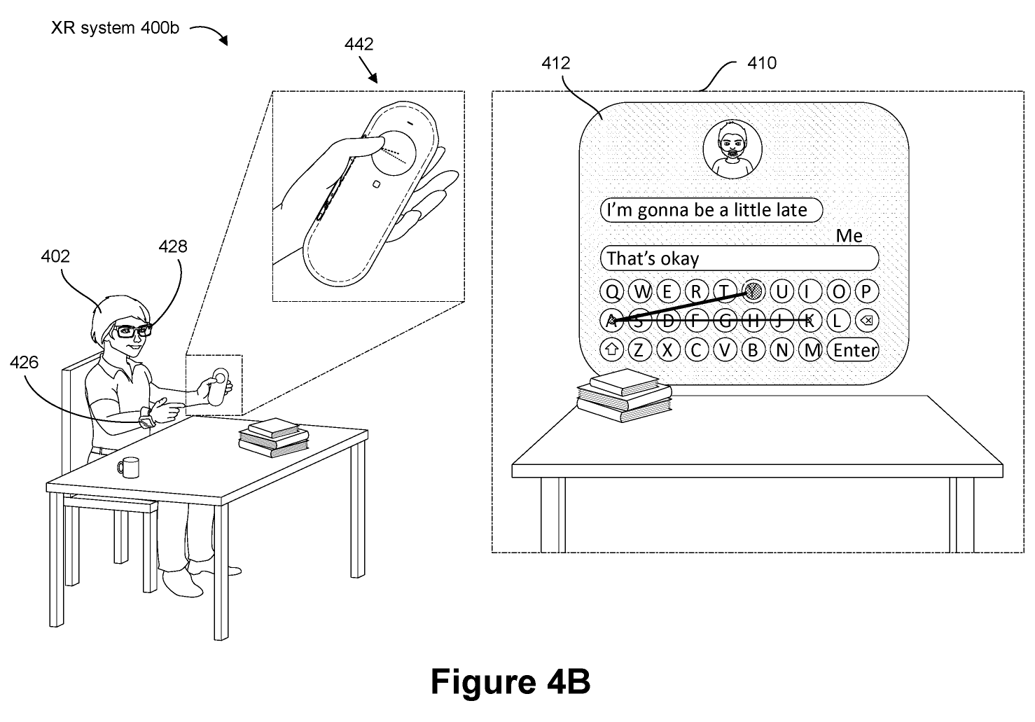

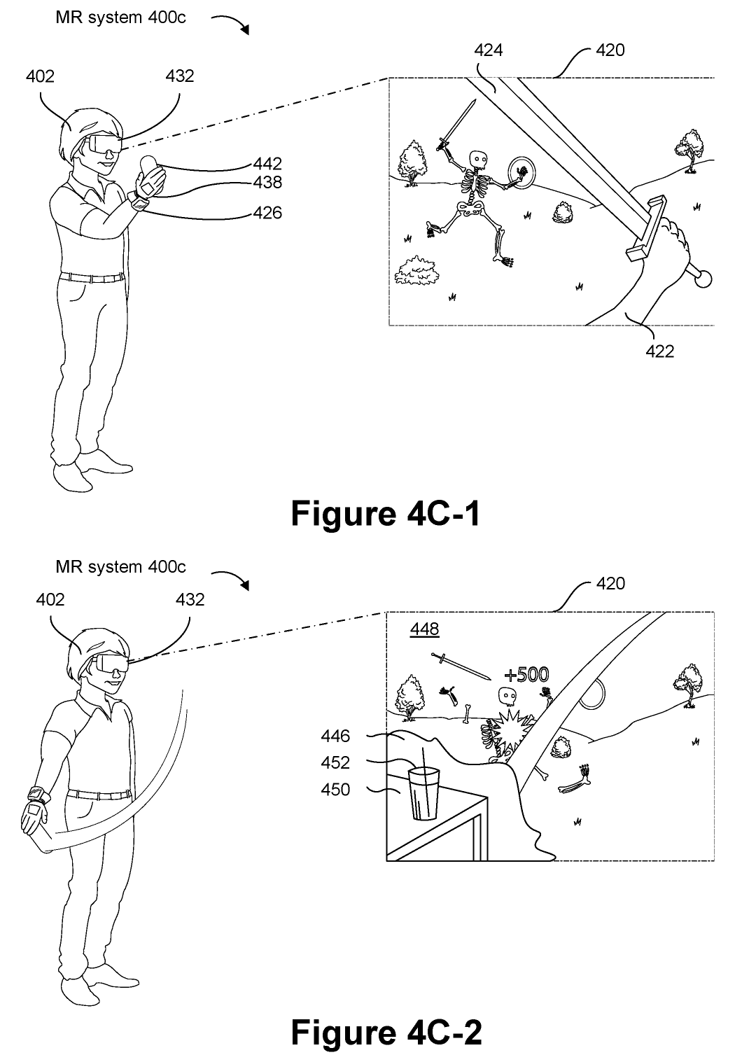

FIGS. 4A 4B, 4C-1, and 4C-2, illustrate example XR systems that include AR and MR systems, in accordance with some embodiments. FIG. 4A shows a first XR system 400a and first example user interactions using a wrist-wearable device 426, a head-wearable device (e.g., AR device 428), and/or a HIPD 442. FIG. 4B shows a second XR system 400b and second example user interactions using a wrist-wearable device 426, AR device 428, and/or an HIPD 442. FIGS. 4C-1 and 4C-2 show a third MR system 400c and third example user interactions using a wrist-wearable device 426, a head-wearable device (e.g., an MR device such as a VR device), and/or an HIPD 442. As the skilled artisan will appreciate upon reading the descriptions provided herein, the above-example AR and MR systems (described in detail below) can perform various functions and/or operations.

The wrist-wearable device 426, the head-wearable devices, and/or the HIPD 442 can communicatively couple via a network 425 (e.g., cellular, near field, Wi-Fi, personal area network, wireless LAN). Additionally, the wrist-wearable device 426, the head-wearable device, and/or the HIPD 442 can also communicatively couple with one or more servers 430, computers 440 (e.g., laptops, computers), mobile devices 450 (e.g., smartphones, tablets), and/or other electronic devices via the network 425 (e.g., cellular, near field, Wi-Fi, personal area network, wireless LAN). Similarly, a smart textile-based garment, when used, can also communicatively couple with the wrist-wearable device 426, the head-wearable device(s), the HIPD 442, the one or more servers 430, the computers 440, the mobile devices 450, and/or other electronic devices via the network 425 to provide inputs.

Turning to FIG. 4A, a user 402 is shown wearing the wrist-wearable device 426 and the AR device 428 and having the HIPD 442 on their desk. The wrist-wearable device 426, the AR device 428, and the HIPD 442 facilitate user interaction with an AR environment. In particular, as shown by the first AR system 400a, the wrist-wearable device 426, the AR device 428, and/or the HIPD 442 cause presentation of one or more avatars 404, digital representations of contacts 406, and virtual objects 408. As discussed below, the user 402 can interact with the one or more avatars 404, digital representations of the contacts 406, and virtual objects 408 via the wrist-wearable device 426, the AR device 428, and/or the HIPD 442. In addition, the user 402 is also able to directly view physical objects in the environment, such as a physical table 429, through transparent lens(es) and waveguide(s) of the AR device 428. Alternatively, an MR device could be used in place of the AR device 428 and a similar user experience can take place, but the user would not be directly viewing physical objects in the environment, such as table 429, and would instead be presented with a virtual reconstruction of the table 429 produced from one or more sensors of the MR device (e.g., an outward facing camera capable of recording the surrounding environment).

The user 402 can use any of the wrist-wearable device 426, the AR device 428 (e.g., through physical inputs at the AR device and/or built-in motion tracking of a user’s extremities), a smart-textile garment, externally mounted extremity tracking device, the HIPD 442 to provide user inputs, etc. For example, the user 402 can perform one or more hand gestures that are detected by the wrist-wearable device 426 (e.g., using one or more EMG sensors and/or IMUs built into the wrist-wearable device) and/or AR device 428 (e.g., using one or more image sensors or cameras) to provide a user input. Alternatively, or additionally, the user 402 can provide a user input via one or more touch surfaces of the wrist-wearable device 426, the AR device 428, and/or the HIPD 442, and/or voice commands captured by a microphone of the wrist-wearable device 426, the AR device 428, and/or the HIPD 442. The wrist-wearable device 426, the AR device 428, and/or the HIPD 442 include an artificially intelligent digital assistant to help the user in providing a user input (e.g., completing a sequence of operations, suggesting different operations or commands, providing reminders, confirming a command). For example, the digital assistant can be invoked through an input occurring at the AR device 428 (e.g., via an input at a temple arm of the AR device 428). In some embodiments, the user 402 can provide a user input via one or more facial gestures and/or facial expressions. For example, cameras of the wrist-wearable device 426, the AR device 428, and/or the HIPD 442 can track the user 402’s eyes for navigating a user interface.

The wrist-wearable device 426, the AR device 428, and/or the HIPD 442 can operate alone or in conjunction to allow the user 402 to interact with the AR environment. In some embodiments, the HIPD 442 is configured to operate as a central hub or control center for the wrist-wearable device 426, the AR device 428, and/or another communicatively coupled device. For example, the user 402 can provide an input to interact with the AR environment at any of the wrist-wearable device 426, the AR device 428, and/or the HIPD 442, and the HIPD 442 can identify one or more back-end and front-end tasks to cause the performance of the requested interaction and distribute instructions to cause the performance of the one or more back-end and front-end tasks at the wrist-wearable device 426, the AR device 428, and/or the HIPD 442. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 442 can perform the back-end tasks and provide the wrist-wearable device 426 and/or the AR device 428 operational data corresponding to the performed back-end tasks such that the wrist-wearable device 426 and/or the AR device 428 can perform the front-end tasks. In this way, the HIPD 442, which has more computational resources and greater thermal headroom than the wrist-wearable device 426 and/or the AR device 428, performs computationally intensive tasks and reduces the computer resource utilization and/or power usage of the wrist-wearable device 426 and/or the AR device 428.

In the example shown by the first AR system 400a, the HIPD 442 identifies one or more back-end tasks and front-end tasks associated with a user request to initiate an AR video call with one or more other users (represented by the avatar 404 and the digital representation of the contact 406) and distributes instructions to cause the performance of the one or more back-end tasks and front-end tasks. In particular, the HIPD 442 performs back-end tasks for processing and/or rendering image data (and other data) associated with the AR video call and provides operational data associated with the performed back-end tasks to the AR device 428 such that the AR device 428 performs front-end tasks for presenting the AR video call (e.g., presenting the avatar 404 and the digital representation of the contact 406).

In some embodiments, the HIPD 442 can operate as a focal or anchor point for causing the presentation of information. This allows the user 402 to be generally aware of where information is presented. For example, as shown in the first AR system 400a, the avatar 404 and the digital representation of the contact 406 are presented above the HIPD 442. In particular, the HIPD 442 and the AR device 428 operate in conjunction to determine a location for presenting the avatar 404 and the digital representation of the contact 406. In some embodiments, information can be presented within a predetermined distance from the HIPD 442 (e.g., within five meters). For example, as shown in the first AR system 400a, virtual object 408 is presented on the desk some distance from the HIPD 442. Similar to the above example, the HIPD 442 and the AR device 428 can operate in conjunction to determine a location for presenting the virtual object 408. Alternatively, in some embodiments, presentation of information is not bound by the HIPD 442. More specifically, the avatar 404, the digital representation of the contact 406, and the virtual object 408 do not have to be presented within a predetermined distance of the HIPD 442. While an AR device 428 is described working with an HIPD, an MR headset can be interacted with in the same way as the AR device 428.

User inputs provided at the wrist-wearable device 426, the AR device 428, and/or the HIPD 442 are coordinated such that the user can use any device to initiate, continue, and/or complete an operation. For example, the user 402 can provide a user input to the AR device 428 to cause the AR device 428 to present the virtual object 408 and, while the virtual object 408 is presented by the AR device 428, the user 402 can provide one or more hand gestures via the wrist-wearable device 426 to interact and/or manipulate the virtual object 408. While an AR device 428 is described working with a wrist-wearable device 426, an MR headset can be interacted with in the same way as the AR device 428.

### Integration of Artificial Intelligence with XR Systems

FIG. 4A illustrates an interaction in which an artificially intelligent virtual assistant can assist in requests made by a user 402. The AI virtual assistant can be used to complete open-ended requests made through natural language inputs by a user 402. For example, in FIG. 4A the user 402 makes an audible request 444 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 4A also illustrates an example neural network 452 used in Artificial Intelligence applications. Uses of Artificial Intelligence (AI) are varied and encompass many different aspects of the devices and systems described herein. AI capabilities cover a diverse range of applications and deepen interactions between the user 402 and user devices (e.g., the AR device 428, an MR device 432, the HIPD 442, the wrist-wearable device 426). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user’s request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 402 can interact with an AI model through natural language inputs captured by a voice sensor, text inputs, or any other input modality that accepts natural language and/or a corresponding voice sensor module. In another instance, input is provided by tracking the eye gaze of a user 402 via a gaze tracker module. Additionally, the AI model can also receive inputs beyond those supplied by a user 402. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors’ data can be retrieved entirely from a single device (e.g., AR device 428) or from multiple devices that are in communication with each other (e.g., a system that includes at least two of an AR device 428, an MR device 432, the HIPD 442, the wrist-wearable device 426, etc.). The AI model can also access additional information (e.g., one or more servers 430, the computers 440, the mobile devices 450, and/or other electronic devices) via a network 425.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 428, an MR device 432, the HIPD 442, the wrist-wearable device 426) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 428, an MR device 432, the HIPD 442, the wrist-wearable device 426), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 442), haptic feedback can provide information to the user 402. An AI model can also use the inputs described above to determine the appropriate modality and device(s) to present content to the user (e.g., a user walking on a busy road can be presented with an audio output instead of a visual output to avoid distracting the user 402).

### Example Augmented Reality Interaction

FIG. 4B shows the user 402 wearing the wrist-wearable device 426 and the AR device 428 and holding the HIPD 442. In the second AR system 400b, the wrist-wearable device 426, the AR device 428, and/or the HIPD 442 are used to receive and/or provide one or more messages to a contact of the user 402. In particular, the wrist-wearable device 426, the AR device 428, and/or the HIPD 442 detect and coordinate one or more user inputs to initiate a messaging application and prepare a response to a received message via the messaging application.

In some embodiments, the user 402 initiates, via a user input, an application on the wrist-wearable device 426, the AR device 428, and/or the HIPD 442 that causes the application to initiate on at least one device. For example, in the second AR system 400b the user 402 performs a hand gesture associated with a command for initiating a messaging application (represented by messaging user interface 412); the wrist-wearable device 426 detects the hand gesture; and, based on a determination that the user 402 is wearing the AR device 428, causes the AR device 428 to present a messaging user interface 412 of the messaging application. The AR device 428 can present the messaging user interface 412 to the user 402 via its display (e.g., as shown by user 402’s field of view 410). In some embodiments, the application is initiated and can be run on the device (e.g., the wrist-wearable device 426, the AR device 428, and/or the HIPD 442) that detects the user input to initiate the application, and the device provides another device operational data to cause the presentation of the messaging application. For example, the wrist-wearable device 426 can detect the user input to initiate a messaging application, initiate and run the messaging application, and provide operational data to the AR device 428 and/or the HIPD 442 to cause presentation of the messaging application. Alternatively, the application can be initiated and run at a device other than the device that detected the user input. For example, the wrist-wearable device 426 can detect the hand gesture associated with initiating the messaging application and cause the HIPD 442 to run the messaging application and coordinate the presentation of the messaging application.

Further, the user 402 can provide a user input provided at the wrist-wearable device 426, the AR device 428, and/or the HIPD 442 to continue and/or complete an operation initiated at another device. For example, after initiating the messaging application via the wrist-wearable device 426 and while the AR device 428 presents the messaging user interface 412, the user 402 can provide an input at the HIPD 442 to prepare a response (e.g., shown by the swipe gesture performed on the HIPD 442). The user 402’s gestures performed on the HIPD 442 can be provided and/or displayed on another device. For example, the user 402’s swipe gestures performed on the HIPD 442 are displayed on a virtual keyboard of the messaging user interface 412 displayed by the AR device 428.

In some embodiments, the wrist-wearable device 426, the AR device 428, the HIPD 442, and/or other communicatively coupled devices can present one or more notifications to the user 402. The notification can be an indication of a new message, an incoming call, an application update, a status update, etc. The user 402 can select the notification via the wrist-wearable device 426, the AR device 428, or the HIPD 442 and cause presentation of an application or operation associated with the notification on at least one device. For example, the user 402 can receive a notification that a message was received at the wrist-wearable device 426, the AR device 428, the HIPD 442, and/or other communicatively coupled device and provide a user input at the wrist-wearable device 426, the AR device 428, and/or the HIPD 442 to review the notification, and the device detecting the user input can cause an application associated with the notification to be initiated and/or presented at the wrist-wearable device 426, the AR device 428, and/or the HIPD 442.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 428 can present to the user 402 game application data and the HIPD 442 can use a controller to provide inputs to the game. Similarly, the user 402 can use the wrist-wearable device 426 to initiate a camera of the AR device 428, and the user can use the wrist-wearable device 426, the AR device 428, and/or the HIPD 442 to manipulate the image capture (e.g., zoom in or out, apply filters) and capture image data.

While an AR device 428 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user’s hand or other suitable surface (e.g., a table, whiteboard). In yet another embodiment, information may also be provided by locally dimming portions of a lens to emphasize portions of the environment in which the user’s attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 4C-1 and 4C-2, the user 402 is shown wearing the wrist-wearable device 426 and an MR device 432 (e.g., a device capable of providing either an entirely VR experience or an MR experience that displays object(s) from a physical environment at a display of the device) and holding the HIPD 442. In the third AR system 400c, the wrist-wearable device 426, the MR device 432, and/or the HIPD 442 are used to interact within an MR environment, such as a VR game or other MR/VR application. While the MR device 432 presents a representation of a VR game (e.g., first MR game environment 420) to the user 402, the wrist-wearable device 426, the MR device 432, and/or the HIPD 442 detect and coordinate one or more user inputs to allow the user 402 to interact with the VR game.

In some embodiments, the user 402 can provide a user input via the wrist-wearable device 426, the MR device 432, and/or the HIPD 442 that causes an action in a corresponding MR environment. For example, the user 402 in the third MR system 400c (shown in FIG. 4C-1) raises the HIPD 442 to prepare for a swing in the first MR game environment 420. The MR device 432, responsive to the user402 raising the HIPD 442, causes the MR representation of the user 422 to perform a similar action (e.g., raise a virtual object, such as a virtual sword 424). In some embodiments, each device uses respective sensor data and/or image data to detect the user input and provide an accurate representation of the user 402’s motion. For example, image sensors (e.g., SLAM cameras or other cameras) of the HIPD 442 can be used to detect a position of the HIPD 442 relative to the user 402’s body such that the virtual object can be positioned appropriately within the first MR game environment 420; sensor data from the wrist-wearable device 426 can be used to detect a velocity at which the user 402 raises the HIPD 442 such that the MR representation of the user 422 and the virtual sword 424 are synchronized with the user 402’s movements; and image sensors of the MR device 432 can be used to represent the user 402’s body, boundary conditions, or real-world objects within the first MR game environment 420.

In FIG. 4C-2, the user 402 performs a downward swing while holding the HIPD 442. The user 402’s downward swing is detected by the wrist-wearable device 426, the MR device 432, and/or the HIPD 442 and a corresponding action is performed in the first MR game environment 420. In some embodiments, the data captured by each device is used to improve the user’s experience within the MR environment. For example, sensor data of the wrist-wearable device 426 can be used to determine a speed and/or force at which the downward swing is performed and image sensors of the HIPD 442 and/or the MR device 432 can be used to determine a location of the swing and how it should be represented in the first MR game environment 420, which, in turn, can be used as inputs for the MR environment (e.g., game mechanics, which can use detected speed, force, locations, and/or aspects of the user 402’s actions to classify a user’s inputs (e.g., user performs a light strike, hard strike, critical strike, glancing strike, miss) or calculate an output (e.g., amount of damage)).

FIG. 4C-2 further illustrates that a portion of the physical environment is reconstructed and displayed at a display of the MR device 432 while the MR game environment 420 is being displayed. In this instance, a reconstruction of the physical environment 446 is displayed in place of a portion of the MR game environment 420 when object(s) in the physical environment are potentially in the path of the user (e.g., a collision with the user and an object in the physical environment are likely). Thus, this example MR game environment 420 includes (i) an immersive VR portion 448 (e.g., an environment that does not have a corollary counterpart in a nearby physical environment) and (ii) a reconstruction of the physical environment 446 (e.g., table 450 and cup 452). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 426, the MR device 432, and/or the HIPD 442 are described as detecting user inputs, in some embodiments, user inputs are detected at a single device (with the single device being responsible for distributing signals to the other devices for performing the user input). For example, the HIPD 442 can operate an application for generating the first MR game environment 420 and provide the MR device 432 with corresponding data for causing the presentation of the first MR game environment 420, as well as detect the user 402’s movements (while holding the HIPD 442) to cause the performance of corresponding actions within the first MR game environment 420. Additionally or alternatively, in some embodiments, operational data (e.g., sensor data, image data, application data, device data, and/or other data) of one or more devices is provided to a single device (e.g., the HIPD 442) to process the operational data and cause respective devices to perform an action associated with processed operational data.

In some embodiments, the user 402 can wear a wrist-wearable device 426, wear an MR device 432, wear smart textile-based garments 438 (e.g., wearable haptic gloves), and/or hold an HIPD 442 device. In this embodiment, the wrist-wearable device 426, the MR device 432, and/or the smart textile-based garments 438 are used to interact within an MR environment (e.g., any AR or MR system described above in reference to FIGS. 4A–4B). While the MR device 432 presents a representation of an MR game (e.g., second MR game environment 420) to the user 402, the wrist-wearable device 426, the MR device 432, and/or the smart textile-based garments 438 detect and coordinate one or more user inputs to allow the user 402 to interact with the MR environment.

In some embodiments, the user 402 can provide a user input via the wrist-wearable device 426, an HIPD 442, the MR device 432, and/or the smart textile-based garments 438 that causes an action in a corresponding MR environment. In some embodiments, each device uses respective sensor data and/or image data to detect the user input and provide an accurate representation of the user 402’s motion. While four different input devices are shown (e.g., a wrist-wearable device 426, an MR device 432, an HIPD 442, and a smart textile-based garment 438) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 438) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user’s experience within the MR environment. Although not shown, the smart textile-based garments 438 can be used in conjunction with an MR device and/or an HIPD 442.

While some experiences are described as occurring on an AR device and other experiences are described as occurring on an MR device, one skilled in the art would appreciate that experiences can be ported over from an MR device to an AR device, and vice versa.

### Other Interactions

While numerous examples are described in this application related to extended-reality environments, one skilled in the art would appreciate that certain interactions may be possible with other devices. For example, a user may interact with a robot (e.g., a humanoid robot, a task specific robot, or other type of robot) to perform tasks inclusive of, leading to, and/or otherwise related to the tasks described herein. In some embodiments, these tasks can be user specific and learned by the robot based on training data supplied by the user and/or from the user's wearable devices (including head-worn and wrist-worn, among others) in accordance with techniques described herein. As one example, this training data can be received from the numerous devices described in this application (e.g., from sensor data and user-specific interactions with head-wearable devices, wrist-wearable devices, intermediary processing devices, or any combination thereof). Other data sources are also conceived outside of the devices described here. For example, AI models for use in a robot can be trained using a blend of user-specific data and non-user specific-aggregate data. The robots may also be able to perform tasks wholly unrelated to extended reality environments, and can be used for performing quality-of-life tasks (e.g., performing chores, completing repetitive operations, etc.). In certain embodiments or circumstances, the techniques and/or devices described herein can be integrated with and/or otherwise performed by the robot.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 4A–4C-2 provided above are intended to augment the description provided in reference to FIGS. 1-3. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A non-transitory, computer-readable storage medium including executable instructions that, when executed by one or more processors, cause the one or more processors to:
while a wrist-wearable device and a calibration device are worn by a user, the wrist-wearable device including one or more photoplethysmography (PPG) sensors and the calibration device including one or more calibration PPG sensors:
while the wrist-wearable device is at a first position on a wrist of the user:
receive first PPG data captured at the one or more PPG sensors and first calibration PPG data captured at the one or more calibration PPG sensors;
generate first physiological data based on the first PPG data and first calibration physiological data based on the first calibration PPG data; and
determine a first placement error based on a comparison between the first PPG data and the first calibration PPG data;
while the wrist-wearable device is at a second position, distinct from the first position, on the wrist of the user:
receive second PPG data captured at the one or more PPG sensors and second calibration PPG data captured at the one or more calibration PPG sensors;
generate second physiological data based on the second PPG data and second calibration physiological data based on the second calibration PPG data; and
determine a second placement error based on a comparison between the second PPG data and the second calibration PPG data; and
in accordance with a determination that the first placement error is less than the second placement error, cause a recommendation to be presented to the user, the recommendation recommending that the user wear the wrist-wearable device at the first position on the wrist of the user.

2. The non-transitory, computer-readable storage medium of claim 1, wherein the executable instructions further cause the one or more processors to:
while the wrist-wearable device and the calibration device are worn by the user:
while the wrist-wearable device is at a third position on the wrist of the user:
receive third PPG data captured at the one or more PPG sensors and third calibration PPG data captured at the one or more calibration PPG sensors;
generate third physiological data based on the third PPG data and third calibration physiological data based on the third calibration PPG data; and
determine a third placement error based on a comparison between the third PPG data and the third calibration PPG data;
while the wrist-wearable device is at a fourth position, distinct from the third position, on the wrist of the user:
receive fourth PPG data captured at the one or more PPG sensors and fourth calibration PPG data captured at the one or more calibration PPG sensors;
generate fourth physiological data based on the fourth PPG data and fourth calibration physiological data based on the fourth calibration PPG data; and
determine a fourth placement error based on a comparison between the fourth PPG data and the fourth PPG data;
while the wrist-wearable device is at a fifth position, distinct from the third position and the fourth position, on the wrist of the user:
receive fifth PPG data captured at the one or more PPG sensors and fifth calibration PPG data captured at the one or more calibration PPG sensors;
generate fifth physiological data based on the fifth PPG data and fifth calibration physiological data based on the fifth calibration PPG data; and
determine a fifth placement error based on a comparison between the fifth PPG data and the fifth PPG data; and
in accordance with a determination that the fifth placement error is less than the third placement error and the fourth placement error, cause another recommendation to be presented to the user, the other recommendation recommending that the user wear the wrist-wearable device at the fifth position on the wrist of the user.

3. The non-transitory, computer-readable storage medium of claim 1, wherein the executable instructions further cause the one or more processors to:
while the wrist-wearable device and the calibration device are worn by the user:
while the wrist-wearable device has a first tightness around the wrist of the user:
receive sixth PPG data captured at the one or more PPG sensors and sixth calibration PPG data captured at the one or more calibration PPG sensors;
generate sixth physiological data based on the sixth PPG data and sixth calibration physiological data based on the sixth calibration PPG data; and
determine a sixth placement error based on a comparison between the sixth PPG data and the sixth calibration PPG data;
while the wrist-wearable device has a second tightness, distinct from the first tightness, around the wrist of the user:
receive seventh PPG data captured at the one or more PPG sensors and seventh calibration PPG data captured at the one or more calibration PPG sensors; and
generate seventh physiological data based on the seventh PPG data and seventh calibration physiological data based on the seventh calibration PPG data; and
determine a seventh placement error based on a comparison between the seventh PPG data and the seventh calibration PPG data; and
in accordance with a determination that the seventh placement error is less than the sixth placement error, cause an additional recommendation to be presented to the user, the additional recommendation recommending that the user wear the wrist-wearable device with the second tightness around the wrist of the user.

4. The non-transitory, computer-readable storage medium of claim 1, wherein the executable instructions further cause the one or more processors to:
while the wrist-wearable device and the calibration device are worn by the user:
while the wrist-wearable device is at a sixth position on the wrist of the user and has a third tightness around the wrist of the user:
receive eighth PPG data captured at the one or more PPG sensors and eighth calibration PPG data captured at the one or more calibration PPG sensors; and
generate eighth physiological data based on the eighth PPG data and eighth calibration physiological data based on the eighth calibration PPG data;
determine an eighth placement error based on a comparison between the eighth PPG data and the eighth calibration PPG data;
while the wrist-wearable device is at a seventh position, distinct from the sixth position, on the wrist of the user and has a fourth tightness, distinct from the third tightness, around the wrist of the user:
receive ninth PPG data captured at the one or more PPG sensors and ninth calibration PPG data captured at the one or more calibration PPG sensors; and
generate ninth physiological data based on the ninth PPG data and ninth calibration physiological data based on the ninth calibration PPG data; and
determine a ninth placement error based on a comparison between the ninth PPG data and the ninth calibration PPG data; and
in accordance with a determination that the eighth placement error is less than the ninth placement error, cause a further recommendation to be presented to the user, the further recommendation recommending that the user wear the wrist-wearable device at the seventh position on the wrist of the user and with the fourth tightness around the wrist of the user.

5. The non-transitory, computer-readable storage medium of claim 1, wherein:
the wrist-wearable device is worn on the wrist of the user; and
the calibration device is worn on a second wrist, distinct from the wrist, of the user.

6. The non-transitory, computer-readable storage medium of claim 5, wherein:
the first position on the wrist on the user and the second position on the wrist of the user are positions that the user finds comfortable for wearing the wrist-wearable device ; and
the calibration device is worn on at a position on the second wrist that is optimal for capturing PPG data.

7. The non-transitory, computer-readable storage medium of claim 6, wherein the executable instructions further cause the one or more processors to:
before receiving the first PPG data and the first calibration PPG data, cause one or more instructions to be presented to the user, the one or more instructions instructing the user to:
fit the calibration device at the position on the second wrist that is optimal for capturing PPG data; and
fit the wrist-wearable device at a position on the wrist on the user that the user finds comfortable for wearing the wrist-wearable device.

8. The non-transitory, computer-readable storage medium of claim 7, wherein the executable instructions further cause the one or more processors to:
before receiving the second PPG data and the second calibration PPG data, cause one or more additional instructions to be presented to the user, the one or more additional instructions instructing the user to fit the wrist-wearable device at another position on the wrist on the user that the user finds comfortable for wearing the wrist-wearable device.

9. The non-transitory, computer-readable storage medium of claim 1, wherein:
the first PPG data is captured at the one or more PPG sensors and the first calibration PPG data is captured at the one or more calibration PPG sensors simultaneously at a first point in time; and
the second PPG data is captured at the one or more PPG sensors and the second calibration PPG data is captured at the one or more calibration PPG sensors simultaneously at a second point in time, distinct from the first point in time.

10. The non-transitory, computer-readable storage medium of claim 1, wherein:
the first physiological data is generated from the first PPG data using one or more pre-trained models;
the first calibration physiological data is generated from the first calibration PPG data using the one or more pre-trained models;
the second physiological data is generated from the second PPG data using the one or more pre-trained models; and
the second calibration physiological data is generated from the second calibration PPG data using the one or more pre-trained models.

11. The non-transitory, computer-readable storage medium of claim 9, wherein the one or more pre-trained models includes one or more of an artificial intelligence (AI) model and a machine-learning (ML) model.

12. The non-transitory, computer-readable storage medium of claim 9, wherein the first physiological data, the first calibration physiological data, the second physiological data, and the second calibration physiological data each include one or more of respective blood pressure data, respective heart rate data, respective blood oxygen saturation data, respective heart rate variability data, and/or respective respiration rate data.

13. The non-transitory, computer-readable storage medium of claim 1, wherein the recommendation is one or more of:
a visual recommendation presented at one or more displays of one or more of the wrist-wearable device, the calibration device, and another device communicatively coupled to the wrist-wearable device; and
an audio recommendation presented at one or more speakers of one or more of the wrist-wearable device, the calibration device, and the other device.

14. The non-transitory, computer-readable storage medium of claim 1, wherein the wrist-wearable device is a smart watch.

15. A system comprising:
a wrist-wearable device including one or more photoplethysmography (PPG) sensors;
a calibration device including one or more calibration PPG sensors;
one or more processors configured to cause the system to:
while the wrist-wearable device and the calibration device are worn by a user:
while the wrist-wearable device is at a first position on a wrist of the user:
receive first PPG data captured at the one or more PPG sensors and first calibration PPG data captured at the one or more calibration PPG sensors; and
generate first physiological data based on the first PPG data and first calibration physiological data based on the first calibration PPG data;
determine a first placement error based on a comparison between the first PPG data and the first calibration PPG data;
while the wrist-wearable device is at a second position, distinct from the first position, on the wrist of the user:
receive second PPG data captured at the one or more PPG sensors and second calibration PPG data captured at the one or more calibration PPG sensors; and
generate second physiological data based on the second PPG data and second calibration physiological data based on the second calibration PPG data; and
determine a second placement error based on a comparison between the second PPG data and the second calibration PPG data; and
in accordance with a determination that the first placement error is less than the second placement error, cause a recommendation to be presented to the user, the recommendation recommending that the user wear the wrist-wearable device at the first position on the wrist of the user.

16. The system of claim 15, wherein the one or more processors are further configured to cause the system to:
while the wrist-wearable device and the calibration device are worn by the user:
while the wrist-wearable device has a first tightness around the wrist of the user:
receive third PPG data captured at the one or more PPG sensors and third calibration PPG data captured at the one or more calibration PPG sensors; and
generate third physiological data based on the third PPG data and third calibration physiological data based on the third calibration PPG data;
determine a third placement error based on a comparison between the third PPG data and the third calibration PPG data;
while the wrist-wearable device has a second tightness, distinct from the first tightness, around the wrist of the user:
receive fourth PPG data captured at the one or more PPG sensors and fourth calibration PPG data captured at the one or more calibration PPG sensors; and
generate fourth physiological data based on the fourth PPG data and fourth calibration physiological data based on the fourth calibration PPG data; and
determine a fourth placement error based on a comparison between the fourth PPG data and the fourth calibration PPG data; and
in accordance with a determination that the fourth placement error is less than the third placement error, cause an additional recommendation to be presented to the user, the additional recommendation recommending that the user wear the wrist-wearable device with the second tightness around the wrist of the user.

17. The system of claim 15, wherein:
the first position on the wrist on the user and the second position on the wrist of the user are positions that the user finds comfortable for wearing the wrist-wearable device; and
the calibration device is worn on at a position on a second wrist, distinct from the wrist, that is optimal for capturing PPG data.

18. A method comprising:
while a wrist-wearable device and a calibration device are worn by a user, the wrist-wearable device including one or more photoplethysmography (PPG) sensors and the calibration device including one or more calibration PPG sensors:
while the wrist-wearable device is at a first position on a wrist of the user:
capturing first PPG data at the one or more PPG sensors and first calibration PPG data at the one or more calibration PPG sensors; and
generating first physiological data based on the first PPG data and first calibration physiological data based on the first calibration PPG data;
determining a first placement error based on a comparison between the first PPG data and the first calibration PPG data;
while the wrist-wearable device is at a second position, distinct from the first position, on the wrist of the user:
capturing second PPG data at the one or more PPG sensors and second calibration PPG data at the one or more calibration PPG sensors; and
generating second physiological data based on the second PPG data and second calibration physiological data based on the second calibration PPG data; and
determining a second placement error based on a comparison between the second PPG data and the second calibration PPG data; and
in accordance with a determination that the first placement error is less than the second placement error, presenting a recommendation to the user, the recommendation recommending that the user wear the wrist-wearable device at the first position on the wrist of the user.

19. The method of claim 18, further comprising:
while the wrist-wearable device and the calibration device are worn by the user:
while the wrist-wearable device and the calibration device are worn by the user:
while the wrist-wearable device has a first tightness around the wrist of the user:
capturing third PPG data at the one or more PPG sensors and third calibration PPG data at the one or more calibration PPG sensors; and
generating third physiological data based on the third PPG data and third calibration physiological data based on the third calibration PPG data;
determining a third placement error based on a comparison between the third PPG data and the third calibration PPG data;
while the wrist-wearable device has a second tightness, distinct from the first tightness, around the wrist of the user:
capturing fourth PPG data at the one or more PPG sensors and fourth calibration PPG data at the one or more calibration PPG sensors; and
generating fourth physiological data based on the fourth PPG data and fourth calibration physiological data based on the fourth calibration PPG data; and
determining a fourth placement error based on a comparison between the fourth PPG data and the fourth calibration PPG data; and
in accordance with a determination that the fourth placement error is less than the third placement error, presenting an additional recommendation to the user, the additional recommendation recommending that the user wear the wrist-wearable device with the second tightness around the wrist of the user.

20. The method of claim 18, wherein:
the first position on the wrist on the user and the second position on the wrist of the user are positions that the user finds comfortable for wearing the wrist-wearable device; and
the calibration device is worn on at a position on a second wrist, distinct from the wrist, that is optimal for capturing PPG data.
